(19) United States
(12) Patent Application Publication
(10) Pub. No.: US 2019/0150721 A1
(43) Pub. Date: May 23, 2019

(54) HOMOGENOUS ILLUMINATION OF FIBER OPTIC DEVICES

(71) Applicant: Cook Medical Technologies LLC, Bloomington, IN (US)

(72) Inventors: Qing Qing Wang, Bloomington, IN (US); Ralf Spindler, Bloomington, IN (US)

(73) Assignee: Cook Medical Technologies LLC, Bloomington, IN (US)

(21) Appl. No.: 16/193,785

(22) Filed: Nov. 16, 2018

Related U.S. Application Data
(60) Provisional application No. 62/588,718, filed on Nov. 20, 2017.

(51) Int. Cl. A61B 1/07

(52) U.S. Cl. CPC A61B 1/07; A61B 2090/306

(57) ABSTRACT

A catheter system for trans-illumination of a surgical area may include a catheter tube having a distal end and a proximal end. The proximal end may include a non-transparent portion and the distal end may include a transparent fiber optic portion. The transparent fiber optic portion may include a fiber optic capable of transmitting light, a plurality of grain indentations having a depth below the surface of the fiber optic portion that communicate light through the outer surface of the fiber optic portion, and reflective coatings. The plurality of grain indentations may increase in size or density, or increase by both size and density, along the length of the fiber optic to provide for a homogenous scattering of light along the fiber optic. The first and second reflective coatings may absorb or reflect light and be shaped to further facilitate homogeneous light scattering along the fiber optic.

### CROSS-REFERENCE TO RELATED APPLICATIONS

This application claims priority from U.S. Provisional Application No. 62/588,718, filed on Nov. 20, 2017, the entirety of which is hereby fully incorporated by reference herein.

### TECHNICAL FIELD

This disclosure relates to devices providing for the trans-illumination of a surgical area during a surgical procedure, particularly during laparoscopic procedures.

### BACKGROUND

Laparoscopic treatment is a widely used modern surgical procedure in which an incision on a patient is minimized, with an incision usually being 0.5-1.5 cm in length. Laparoscopic treatment allows for the use of fiber optics and miniature camera systems during surgical procedures, and common laparoscopic procedures include hernia repairs, gastric bypass, bowel resection, and organ removal.

An illuminating catheter is a fiber optic device that is used to provide trans-illumination of a surgical area during laparoscopic procedures. The illuminating catheter also helps to identify and minimize potential for trauma during surgical procedures. Accordingly, it is desirable that illuminating catheters provide homogeneous illumination along the entire length of the catheter, or, in other words, homogenous scattering of light along the length of the catheter.

Light scattering involves the deflection of light in a transmission medium. Light is technically known as an electromagnetic (“EM”) wave with a wavelength from about 0.3 to 30 microns, including visible wavelengths from 0.38 to 0.78 microns, and those wavelengths, such as ultraviolet and infrared, that are visible using various optical techniques. Blue light, for example, has a visible wavelength of about 0.475 microns, while red light a wavelength of about 0.65 microns. White light is a mixture of colors in the visible wavelength range, while black is a total absence of light. Fiber optic cables are a type of optical fiber commonly used as a transmission medium for light. Optical fibers usually include a fiber core and a fiber cladding that can guide a lightwave and is usually cylindrical in shape. The fiber relies upon internal reflection to transmit light along its axial length, with light entering one end of the fiber at an initial intensity and emerging from the opposite end with intensity losses dependent upon length, absorption, scattering, and other factors. Light intensity is often referred to as luminous intensity, which is measured as the candela (cd). Intensity used with respect to illuminating devices may range from a fraction of a cd to about 100 cd or higher, depending on the light source used.

There are several types of light scattering modes known in the art, including Rayleigh scattering. Rayleigh scattering involves the scattering of a lightwave being transmitted through a medium, such as a fiber optic, due to the atomic or molecular structure of the material and variations in the structure as a function of distance. For example, as light travels through a fiber optic, scattering loss may occur, which is a loss of power of the EM wave due to random reflections and deflections of the waves caused by the material elements in the fiber optic as well as by impurities, imbedded particles, and inclusions. Scattering loss varies as the reciprocal of the fourth power of the wavelength. The transparency of a material may also affect the amount of light scattered.

It is also desirable to have an illuminating catheter which is compatible with high-power light sources. Common spectral microscopy light sources include tungsten-halogen, mercury, xenon, and metal halide light sources. The Rayleigh scattering intensity will vary depending on the particular light source, as commonly understood in the art. For example, with a xenon light source, mainly low wavelengths are scattered into a patient's tissue (e.g., 0.3 microns).

### BRIEF SUMMARY

A catheter system for trans-illumination of a surgical area may include a catheter tube having a distal end and a proximal end. The proximal end may include a non-transparent portion and the distal end may include a transparent fiber optic portion. The transparent fiber optic portion may include a fiber optic capable of transmitting light, a proximal end, a distal end, a distal tip, a surface that establishes an outer circumference of the fiber optic portion, a plurality of grain indentations having a depth below the surface of the fiber optic portion that communicate light through the outer surface of the fiber optic portion, and first and second coatings. The plurality of grain indentations may increase in size or density, or by both size and density, between neighboring indentations to provide for a homogenous scattering of light along the length of the fiber optic. The first and second coatings may absorb or reflect light and be shaped to further facilitate homogeneous light scattering along the fiber optic. The plurality of grain indentations and the first coating may be disposed along the surface of the transparent fiber optic portion from a first position proximate to the proximal end of the transparent fiber optic portion to a second position proximate to the distal end of the transparent fiber optic portion, and a size or density of the grain indentations increases in a direction from the first position to the second position. The second coating may be disposed at the distal tip of the transparent fiber optic portion.

In one embodiment, a catheter system for trans-illumination of a surgical area may include a catheter tube having a distal end and a proximal end. The proximal end may include a non-transparent portion and the distal end may include a transparent fiber optic portion. The transparent fiber optic portion may include a fiber optic capable of transmitting light, a proximal end, a distal end, a surface that establishes an outer circumference of the fiber optic portion, and a plurality of grain indentations having a depth below the surface of the fiber optic portion that communicate light through the outer surface of the fiber optic portion. The plurality of grain indentations may be disposed along the surface of the transparent fiber optic portion from a first position proximate to the proximal end of the transparent fiber optic portion to a second position proximate to the distal end of the transparent fiber optic portion, and a size of the grain indentations increases in a direction from the first position to the second position. The grain indentations may increase in size between neighboring indentations in various ways, such as an increase in a groupwise manner or a continuous increase, which provides for a homogenous scattering of light along the length of the fiber optic. The plurality of grain indentations may be a repeating pattern along the surface from the first position to the second position. The repeating pattern may include individual grain indentations, or the grain indentations may be connected.

In another embodiment, a catheter system for trans-illumination of a surgical area may include a catheter tube having a distal end and a proximal end. The proximal end may include a non-transparent portion and the distal end may include a transparent fiber optic portion. The transparent fiber optic portion may include a fiber optic capable of transmitting light, a proximal end, a distal end, a surface that establishes an outer circumference of the fiber optic portion, and a plurality of grain indentations having a depth below the surface of the fiber optic portion that communicate light through the outer surface of the fiber optic portion. The plurality of grain indentations may be disposed along the surface of the transparent fiber optic portion from a first position proximate to the proximal end of the transparent fiber optic portion to a second position proximate to the distal end of the transparent fiber optic portion, and a density of the grain indentations increases in a direction from the first position to the second position. The grain indentations may increase in density between neighboring indentations in various ways, such as in a groupwise manner or a continuous increase, which provides for a homogenous scattering of light along the length of the fiber optic. The plurality of grain indentations may be a repeating pattern along the surface from the first position to the second position. The repeating pattern may include individual grain indentations, or the grain indentations may be connected.

In another embodiment, a catheter system for trans-illumination of a surgical area may include a catheter tube having a distal end and a proximal end. The proximal end may include a non-transparent portion and the distal end may include a transparent fiber optic portion. The transparent fiber optic portion may include a fiber optic capable of transmitting light, a proximal end, a distal end, a distal tip, a surface that establishes an outer circumference of the fiber optic portion, a plurality of grain indentations having a depth below the surface of the fiber optic portion that communicate light through the outer surface of the fiber optic portion, and first and second coatings. The plurality of grain indentations and the first coating may be disposed along the surface of the transparent fiber optic portion from a first position proximate to the proximal end of the transparent fiber optic portion to a second position proximate to the distal end of the transparent fiber optic portion, and a size, density, or a combination of both size and density, of the grain indentations increases in a direction from the first position to the second position. The second coating may be disposed at the distal tip of the transparent fiber optic portion. The grain indentations may increase in size or density, or in both size and density, between neighboring indentations in various ways, such as an increase in a groupwise manner or a continuous increase, which provides for a homogenous scattering of light along the length of the fiber optic. The first and second coatings may absorb or reflect light and be shaped to further facilitate homogeneous light scattering along the fiber optic. The plurality of grain indentations may be a repeating pattern along the surface from the first position to the second position. The repeating pattern may include individual grain indentations, or the grain indentations may be connected.

Other systems, methods, features, and advantages of the invention will be, or will become apparent to, one with ordinary skill in the art upon examination of the following figures and detailed description. It is intended that all such additional systems, methods, features and advantages be within the scope of the invention, and be encompassed by the following claims.

### BRIEF DESCRIPTION OF THE DRAWINGS

The accompanying drawings are included to provide a further understanding of the claims, are incorporated in, and constitute a part of this specification. The detailed description and illustrated examples described serve to explain the principles defined by the claims.

FIG. 1 is an orthogonal view of an embodiment of the catheter system with an increase in grain size along the length of the fiber optic from a first to a second position, depicting schematically homogenous scattering of light along the length of the fiber optic.

FIG. 2 is an orthogonal view of an embodiment of the catheter system with an increase in grain density along the length of the fiber optic from a first to a second position, depicting schematically homogenous scattering of light along the length of the fiber optic.

FIG. 3 is an orthogonal view of an embodiment of the catheter system with a constant grain size and density along the length of the fiber optic with a first surface coating along the length of the fiber optic and a second surface coating at the distal tip of the fiber optic, the grain indentations and surface coatings depicting schematically homogenous scattering of light along the length of the fiber optic.

FIG. 4a is an enlarged orthogonal view of a plurality of grain indentations having spherical shapes.

FIG. 4b is an enlarged orthogonal view of a plurality of grain indentations having diamond shapes.

FIG. 4c is an enlarged orthogonal view of a plurality of grain indentations having amorphous shapes

FIG. 5a is a perspective view of a single spherical grain indentation having a depth below the surface of the fiber optic portion equal to the diameter of the spherical grain indentation.

FIG. 5b is a perspective view of a single spherical grain indentation having a depth below the surface of the fiber optic portion less than the diameter of the spherical grain indentation.

FIG. 6a is an orthogonal view of an embodiment of the catheter system schematically depicting connected ring-shaped grain indentations along the length of the fiber optic.

FIG. 6b is an orthogonal view of an embodiment of the catheter system schematically depicting connected helical-shaped grain indentations along the length of the fiber optic.

FIG. 6c is an orthogonal view of an embodiment of the catheter system schematically depicting connected amorphous-shaped grain indentations along the length of the fiber optic.

FIG. 7 is an exploded view of an embodiment of the catheter system wherein the transparent fiber optic portion and the proximal end of the catheter tube are detachably connected.

FIG. 8 is an orthogonal view of an embodiment of the catheter system wherein a light source may supply light to the transparent fiber optic portion of the catheter system and be detachably connected to the proximal end of the catheter tube, and wherein the scattered light intensity per grain plurality group is schematically depicted along the length of the transparent fiber optic portion.

FIGS. 9a and 9b graphically depict FIG. 8's ratio of scattered light intensity per grain plurality group on an X-Y axis.

### DETAILED DESCRIPTION OF THE INVENTION

The embodiments described in this disclosure will be discussed generally in relation to the use of catheter devices providing for the trans-illumination of a surgical area during a laparoscopic surgical procedure, but the disclosure is not so limited and may be applied to the use of other medical devices in other procedures other than laparoscopic procedures.

In the present application, the term “proximal” refers to a direction that is generally closest to the operator of the device during a medical procedure, while the term “distal” refers to a direction that is furthest from the operator of the device. As used herein, “about” and “substantially” mean any deviation within 5 to 10 percent, plus or minus, the recited value.

The present catheter system operates to allow for trans-illumination of a surgical area. In particular, the catheter system may provide for a catheter tube having a transparent fiber optic portion including a fiber optic cable capable of transmitting light during laparoscopic procedures which helps to identify and minimize potential for trauma. A specific pattern of indents, or grain indentations, may be placed along or below the surface of the fiber optic to optimize the homogeneity of the light transmitted along the length of the fiber optic cable and improve the heat compatibility of the catheter system with high-power light sources. The grain indentations may be placed in a groupwise manner along the length of the fiber optic, and may vary in size, density, or a combination of size and density.

One or more coatings may also be placed along the surface or at a distal tip of the fiber optic to absorb or, preferably, reflect light. The shape of the coatings may be designed in such a way to further facilitate homogenous light distribution along the length of the fiber optic.

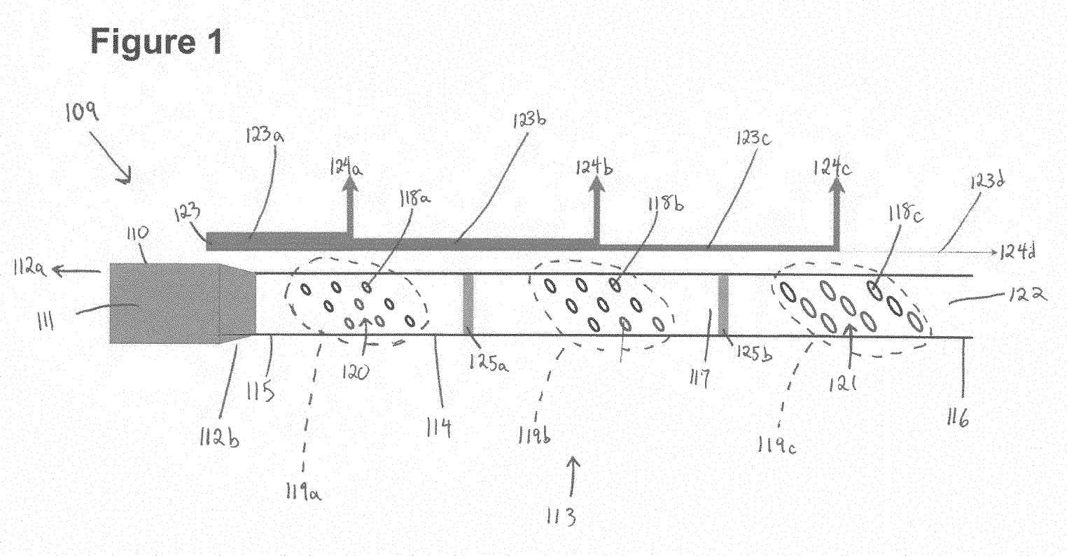

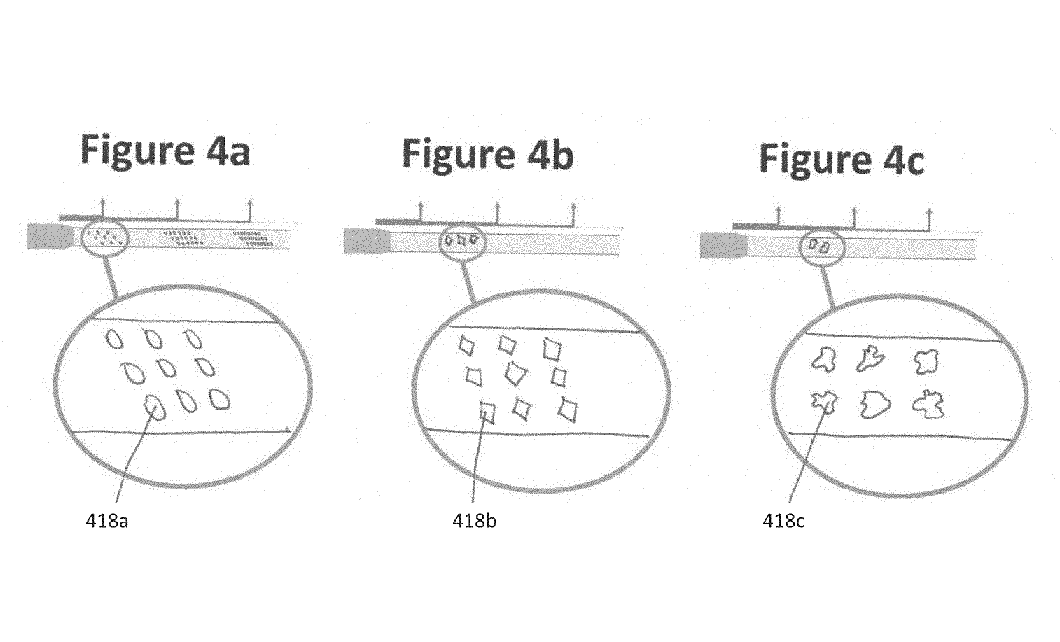

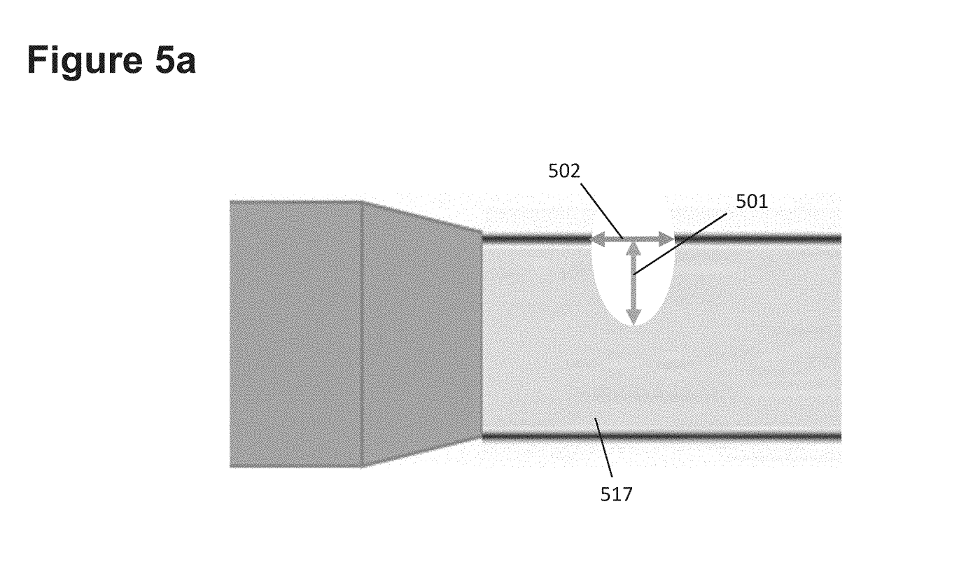

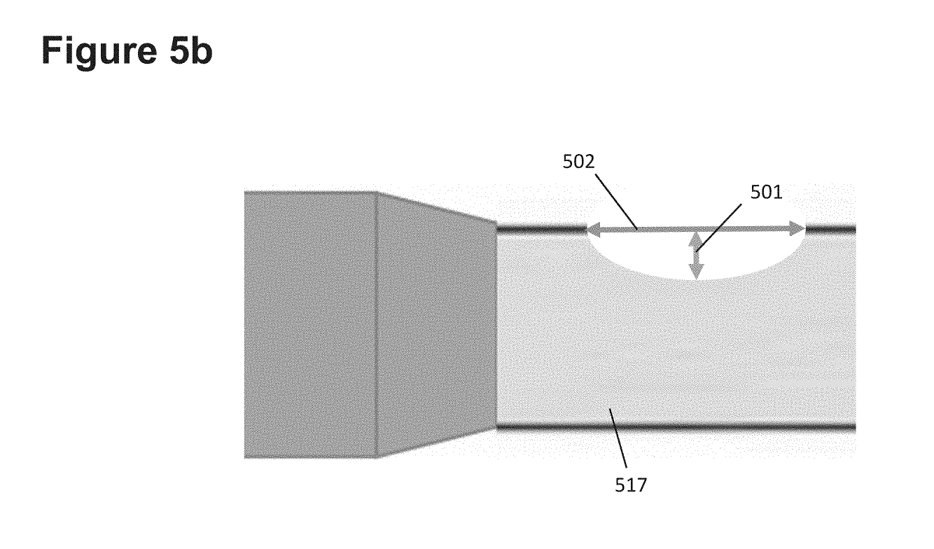

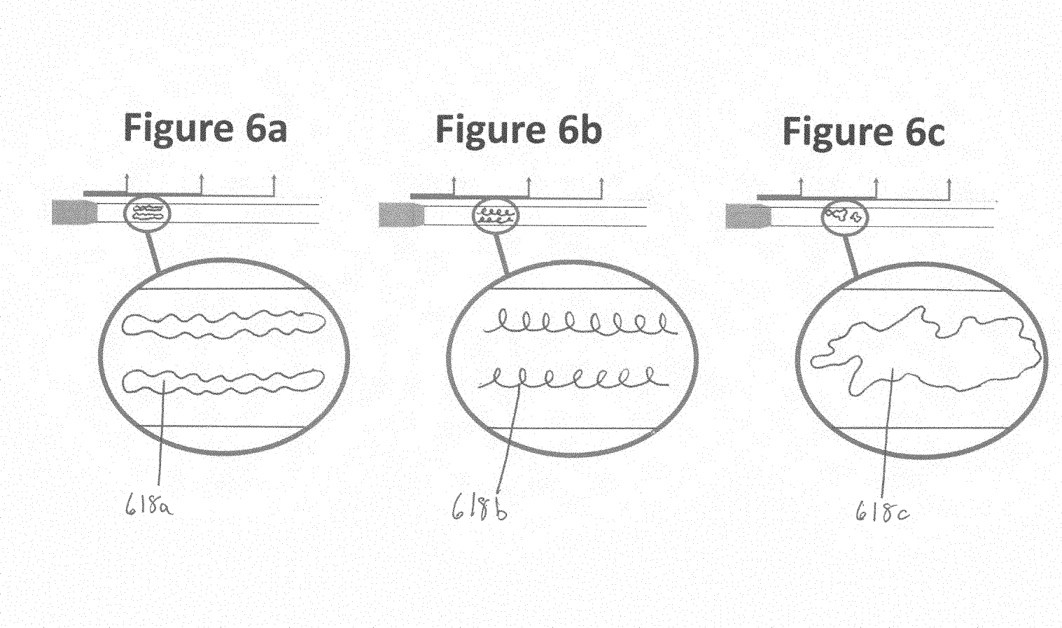

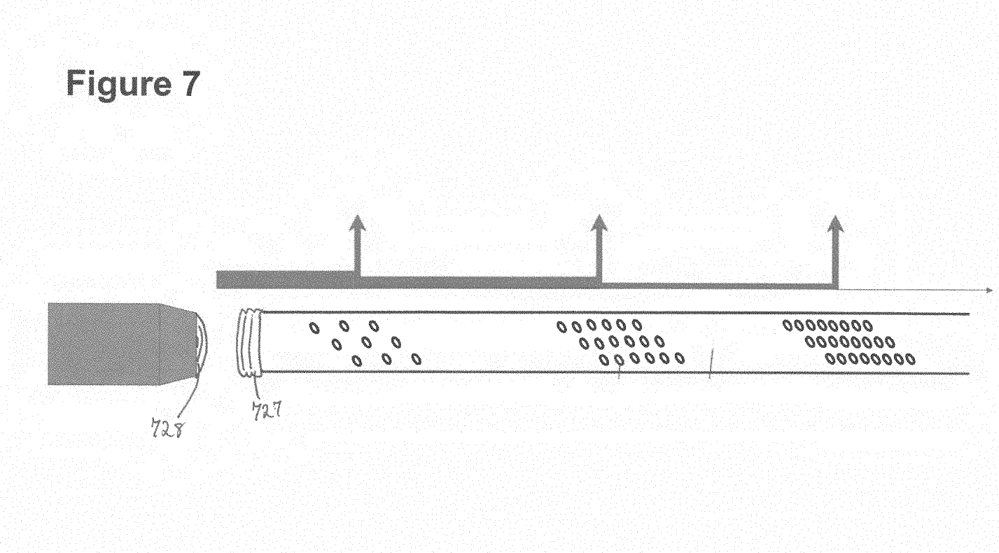

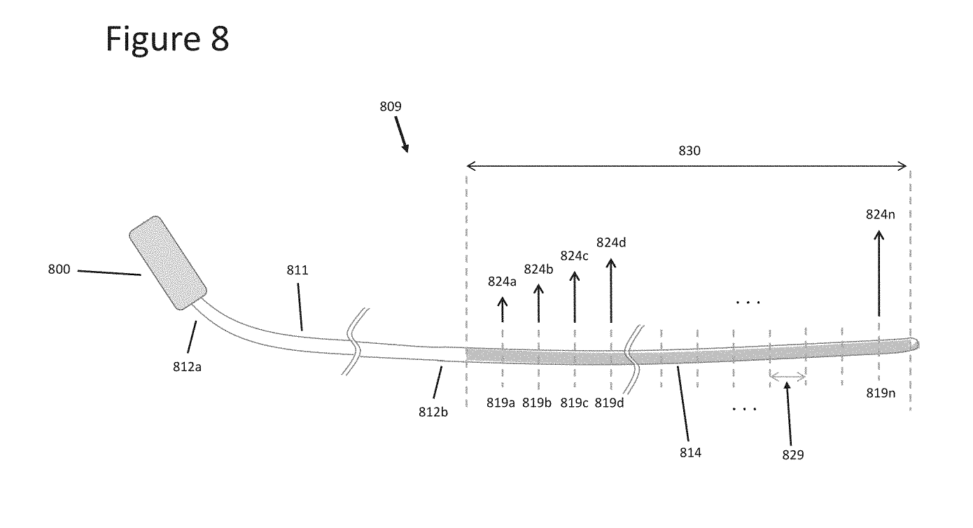

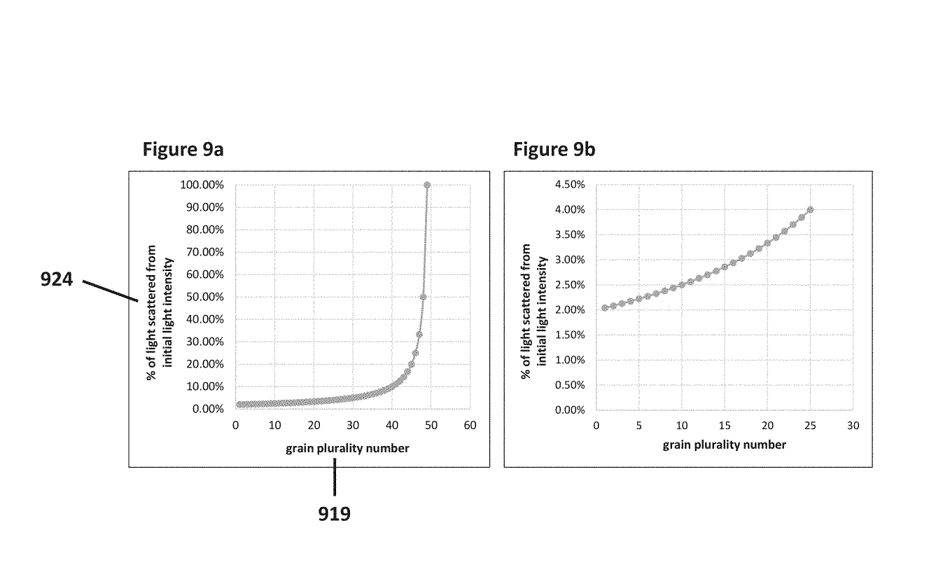

As described more fully below, FIGS. 1-3 illustrate some embodiments of the catheter system. FIGS. 4a-4c and FIGS. 6a-6c illustrate various shapes of the grain indentations. FIGS. 5a-5b illustrate the parameters by which to measure the size of a grain indentation. FIG. 7 illustrates a catheter system whereby the transparent fiber optic portion may be detachably connected to the proximal end of the catheter tube. FIG. 8 illustrates a catheter system having a light source to supply light to the fiber optic portion, the light source being detachably connected to the proximal end of the catheter tube. FIGS. 9a and 9b graphically depict FIG. 8's ratio of scattered light intensity per grain plurality group on an X-Y axis.

For the sake of brevity, like components are depicted with the same element numbers in various embodiments and the reader is referred to the description of those elements in related elements. Elements that share similar features are designated with the same tenths and hundredths place with differing numbers in the hundreds place (e.g. 102, 202, 302, etc.).

FIG. 1 shows an orthogonal view of the catheter system 109 with catheter tube 110, fiber optic 117, and grain indentations 118. Catheter tube 110 may be elongated and flexible, and includes a non-transparent portion 111 at its proximal end and a transparent fiber optic portion 114 at its distal end. The non-transparent portion 111 of the catheter tube 110 may additionally include a proximal end 112a, and a distal end 112b. Transparent fiber optic portion 114 is connected to the distal end 112b of the non-transparent portion 111.

The transparent fiber optic portion 114 may include a fiber optic cable 117 capable of transmitting light 123 with an initial light intensity 123a, a proximal end 115, a distal end 116, a surface 122 that establishes an outer circumference of the fiber optic portion, and a plurality of grain indentations 119a, 119b, and 119c that communicate initial light intensity 123a through the outer surface 122 of the fiber optic portion 114. In some embodiments, a coating or sheath may be disposed over the fiber optic portion to add additional characteristics to the fiber optic portion, such as additional protection of the fiber optic.

Each plurality of grain indentions 119 may include of a group of individual grain indentations 118. Grain indentations 118 may be made upon or below the surface 122 of fiber optic 117 using conventional methods known to those of ordinary skill in the art, including sandblasting and crimping. In one embodiment, it may be a manufacturing preference to have some amount of space between each plurality of grain indentation 119. Each individual grain indentation 118 may include a depth 501 and diameter 502. The depth 501 of the grain indentations may be from 10 to 100 microns, and the diameter 502 of the grain indentation may be from 10 to 100 microns. Grain indentations 118 may also take the form of various shapes, as schematically depicted in FIGS. 4a-4c, including a spherical grain indentation 418a, a diamond grain indentation 418b, and an orientation with an arbitrary shape, such as that schematically depicted in 418c, or other shapes. The size, shape, and pattern of the grain may be varied to obtain a desired scattered light spectrum along the length of the fiber optic. This result is primarily due to an optical grating effect, as specially constructed reflector patterns created in a segment of an optical fiber may reflect specific wavelengths of light and transmit others.

In some embodiments, the individual grain indentations 118 may increase in size along the length of the fiber optic 117 with each plurality of grain indentations 119, such as schematically depicted by grain indentations 118a, 118b, and 118c. A plurality of grain indentations 119 may be disposed along the surface 122 of the transparent fiber optic portion 114 from a first position 120 proximate to the proximal end 115 of the transparent fiber optic portion 114 to a second position 121 proximate to the distal end 116 of the transparent fiber optic portion 114, and a size of the individual grain indentations 118 may increase in a direction from the first position 120 to the second position 121, as shown in FIG. 1.

The size of grain indentations 118 of grain pluralities 119 may increase along the length of the fiber optic 117 in various ways. For example, grain indentations 118 may continuously increase in size as schematically depicted in FIG. 1 from the first position 120 to the second position 121, or, in other embodiments, the grain indentations 118 may increase in size in a groupwise manner between neighboring indentations, such as schematically depicted in FIG. 1.

The plurality of grains 119 may also be arranged in various patterns, including the repeating rhombus-shaped pattern of three-by-three grain indentations schematically depicted in FIG. 1. Each repeating pattern may include individual grain indentations 118, or may include connected grain indentations 618 as further described and depicted below in FIGS. 6a-6c.

The increase in size of grain indentations 118 from the first position 120 to the second position 121 facilitates homogeneous light scattering along the length of the fiber optic 117 because more light 123 is scattered as the size of grain indentations 118 increases and the amount of light 123 traveling through fiber optic 117 decreases along fiber optic 117's length as some light 123 leaves the fiber optic due to scattering.

The degree of light intensity 123a scattered may be affected and optimized by various factors, including the degree of transparency of the fiber optic 117, the size of grains 118 in each plurality of grains 119, the number of plurality of grains 119 along the length of the fiber optic 117, and the particular pattern of grains 118 in each plurality of grains 119. Upon a thorough review of this specification, one of ordinary skill in the art will understand how to optimize the light scattering without undue experimentation.

As shown in FIG. 1, for example, initial light intensity 123a is scattered in about equal percentages, e.g., scattered light portions 124a, 124b, and 124c, at each plurality of grains 119a, 119b, and 119c, respectively, as a result of the pattern and increase in size of grain indentations 118 along the length of fiber optic 117 from the first position 120 to the second position 121. In particular, because grains 118a are smaller than grains 118b, grains 118a will scatter less light than grains 118b for the same amount of incident light that approaches each representative grain, and similarly, grains 118b, which are smaller than grains 118c, will scatter less light than grains 118c for the same amount of incident light that approaches each representative grain.

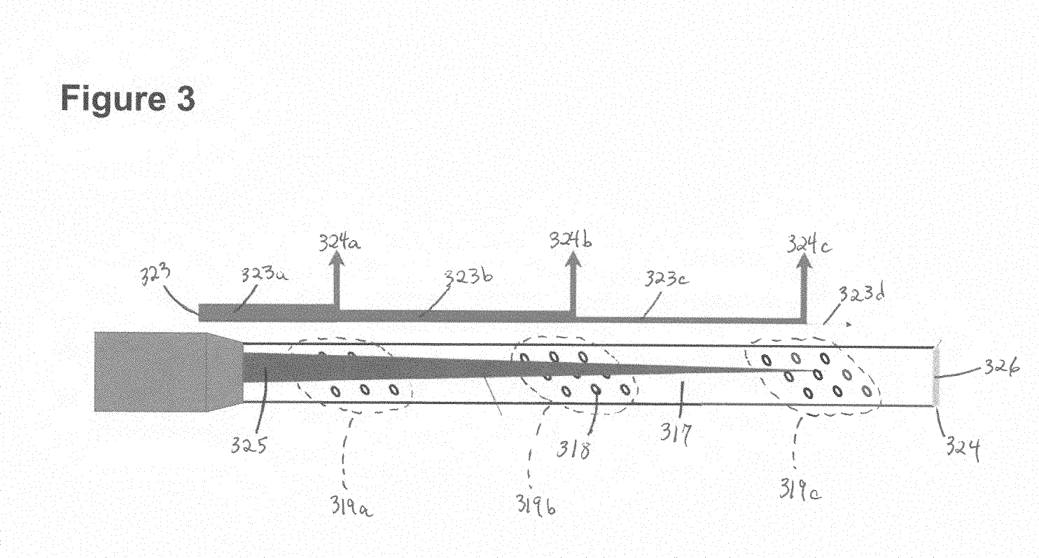

This concept is schematically depicted in FIG. 1, where about 30 percent of initial light intensity 123a is reflected through fiber optic 117 at scattered light portion 124a as a result of the light scattered by the size and pattern of grain plurality 119a. About another 30 percent of initial light intensity 123a is reflected through fiber optic 117 at scattered light portion 124b as a result of the light scattered by the size and pattern of grain plurality 119b, grain plurality 119b scattering more light than grain plurality 119a because grains 118b are larger than grains 118a. And yet about another 30 percent of initial light intensity 123a is reflected through fiber optic 117 at scattered light portion 124c as a result of the light scattered by the size and pattern of grain plurality 119c, grain plurality 119c scattering more light than both grain pluralities 119a and 119b because grains 118c are larger than both grains 118b and 118a. Scattered light portion 124d may represent a residual amount of light intensity emitted at the distal end 116 of the fiber optic 117, and may additionally be reflected proximally back into the fiber optic 117 to additionally facilitate homogenous illumination of fiber optic 117 as further explained below and schematically depicted in FIG. 3.

Grain size generally refers to the grain's volume, which may be determined by at least the grain's diameter on the outer surface of the fiber optic and its depth below the surface of the fiber optic. FIG. 5a is a perspective view of a single spherical grain indentation having a depth 501 below the surface of the fiber optic portion equal to the diameter 502 of the spherical grain indentation. FIG. 5b is a perspective view of a single spherical grain indentation having a depth below the surface of the fiber optic portion less than the diameter of the spherical grain indentation. Generally, the depth of a grain indentation should not be deeper than the grain indentation's diameter (e.g., less than 10 micrometers) to preserve the mechanical properties of the fiber optic; however, this general rule may vary depending on the fiber optic material. Furthermore, a 3-D laser engraving process of the grain indentations onto or below the surface of the fiber optic may be used to create defined optical impurities below the surface of the fiber optic.

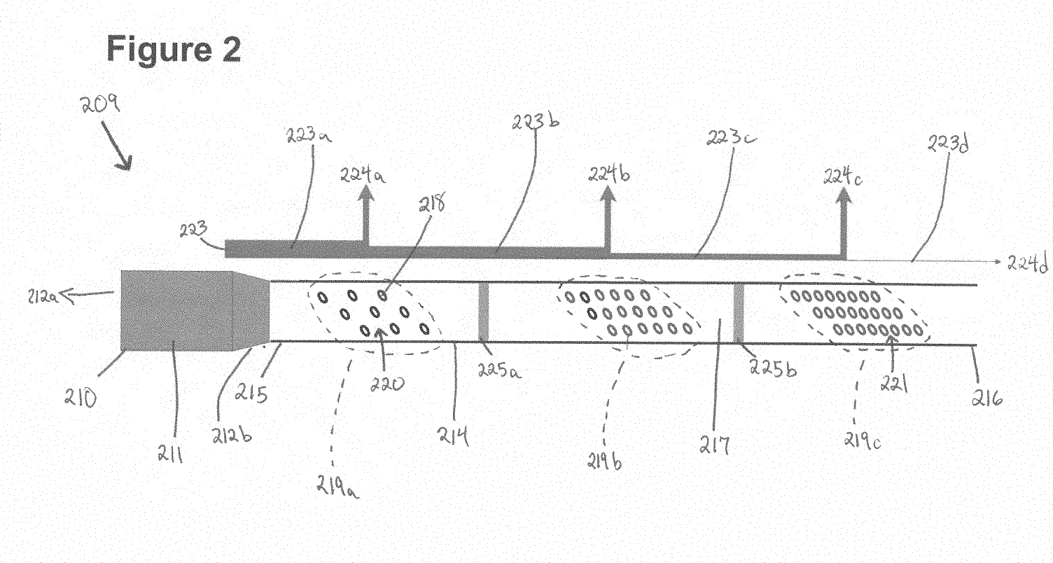

In another embodiment, the catheter system may have one or both of radiopaque 125a and non-radiopaque 125b markings along the surface of the transparent fiber optic portion, as schematically depicted in FIGS. 1 and 2.

FIG. 2 presents a similar embodiment as FIG. 1. As shown in FIG. 2, however, homogeneous light scattering along the length of the fiber optic 217 is facilitated by an increase in density of individual grain indentations 218 in each grain plurality 219 from the first position of grain density to the second position of grain density. While the size of each individual grain indentation 218 remains constant in each grain plurality 219, the increase in grain density from the first position 220 to the second position 221 results in the homogeneous reflection of light along fiber optic 217 from the first position 220 to second position 221 because more light 223 is scattered as the number, or density, of grain indentations 218 is increased. For example, initial light intensity 223a is scattered in about equal percentages, e.g., scattered light portions 224a, 224b, and 224c, at each grain plurality 219a, 219b, and 219c, respectively, as a result of the pattern and increase in density of grain indentations 218 in grain pluralities 219 along the length of fiber optic 217 from the first position 220 to the second position 221.

In particular, because grain plurality 219a has a smaller density of grain indentations 218 than grain plurality 219b, grain plurality 219a will scatter less light than grain plurality 219b for the same amount of incident light that approaches each representative grain, and similarly, grain plurality 219b, which has a smaller density of grain indentations 218 than grain plurality 219c, will scatter less light than grain plurality 219c for the same amount of incident light that approaches each representative grain. By example, this concept is schematically depicted in FIG. 2, where about 30 percent of the initial light intensity 223a is reflected through fiber optic 117 at scattered light portion 224a as a result of the light scattered by the density and pattern of grain plurality 219a. About another 30 percent of initial light intensity 223a is reflected through fiber optic 117 at scattered light portion 224b as a result of the light scattered by the density and pattern of grain plurality 219b, grain plurality 219b scattering more light than grain plurality 219a due to a higher density of grains 218b in grain plurality 219b than in grain plurality 219a. And yet about another 30 percent of initial light intensity 223a is reflected through fiber optic 217 at scattered light portion 224c as a result of the light scattered by the density and pattern of grains 218 in grain plurality 219c, grain plurality 219c scattering more light than both grain pluralities 219a and 219b due to a higher density of grains 118 in grain plurality 219c than in grain pluralities 219b and 219a. Scattered light portion 224d may represent a residual amount of light intensity emitted at the distal end 216 of the fiber optic 217, and may additionally be reflected proximally back into the fiber optic 217 to additionally facilitate homogenous illumination of fiber optic 217 as further explained below and schematically depicted in FIG. 3.

FIG. 3 presents a similar embodiment as both FIGS. 1 and 2. As shown in FIG. 3, however, homogeneous light scattering along the length of the fiber optic 317 is facilitated by a combination of grain pluralities 319 of constant density along the fiber optic, grain indentations 318 of constant size along the fiber optic, a first coating 325, and a second coating 326. In some embodiments, the coating may be a light reflecting coating. In other embodiments, the coating may be a light absorbing coating. Coating 325 may be disposed upon the grain pluralities 319, and coating 326 may be disposed at a distal tip 324 of the fiber optic 317. Coating 325 may cover various portions of the fiber optic surface and may be disposed in various shapes along the fiber optic 317 from a first position 320 at grain plurality 319a to a second position 321 at grain plurality 319c. For example, coating 325 schematically depicted FIG. 3 is disposed as a triangular shape from the first to second position along the fiber optic 317. Coating 325 may further increase or decrease the amount of light scattered at each light scattering portion, depending in part on the transparency of coating 325, and whether it is configured to absorb, rather than reflect, light 323.

The transparency of the materials used for coating 325, e.g., aluminum, gold, or silver, may determine the amount of light 323 ultimately transmitted through fiber optic 317 after light 323 has been scattered by a plurality of grains 119. For example, while about 30 percent of initial light intensity 323a might be normally scattered by grain plurality 319a at light scattering portion 324a, coating 325 may cover a portion or all of grain plurality 319a, and, depending on the amount of transparency of the material used as coating 325, may reduce the 30 percent of light that would otherwise be scattered at light scattering portion 324a without the coating 325. Second coating 326 may further facilitate homogenous illumination of fiber optic 317 by acting as a mirror at the distal tip 324 of fiber optic 317 to cause reflection of any residual amount of light intensity, schematically depicted as 323d, that is not emitted at the light scattering portions 324a, 324b, and 324c along the fiber optic. Coatings 325 and 326 may be manufactured onto the surface of the fiber optic 317 by conventional methods known to those of ordinary skill in the art, including dip-coating, chemical vapor deposition (“CVD”), and physical vapor deposition (“PVD”).

In some embodiments, the shape of the grain indentations may be varied to obtain a desired scattered light spectrum along the length of the fiber optic. FIGS. 4a, 4b, and 4c schematically depict various shapes of the grain indentations. FIG. 4a is an enlarged orthogonal view of a plurality of grain indentations having spherical shapes. FIG. 4b is an orthogonal view of a grain indentation having a diamond shape. FIG. 4c is an orthogonal view of a grain indentation having an arbitrary shape. In other embodiments, the grain indentations may be connected. FIG. 6 is an orthogonal view of an embodiment of the catheter system schematically depicting connected grain indentations 618 along the length of the fiber optic. Connected grain indentations 618 may be various shapes, including ring shape 618a, helical shape 618b, and arbitrary shape 618c, as shown in FIG. 6. In some embodiments, the device may include only certain grain indentation shapes, such as, e.g., only helical connected indentations, while in other embodiments, the device may include different shapes along its length.

FIG. 7 is an exploded view of an embodiment of the catheter system wherein the transparent fiber optic portion and the proximal end of the catheter tube are detachably connected. In some embodiments, the detachable connection may be via a threaded connection 727 and 728, a press fit connecting, a locking connection, a clamped connection, or the like. In some embodiments, the catheter system may be configured to be releasable after an initial connection, while in other embodiments, the components may be permanently connected.

FIG. 8 is an orthogonal view of an embodiment of the catheter system 809 wherein a light source 800 supplies light to a length 830 of the transparent fiber optic portion 814 of the catheter system. Common spectral microscopy light sources include tungsten-halogen, mercury, xenon, and metal halide light sources. The light source 800 may be detachably connected to the proximal end 812a of the non-transparent portion 811 of the catheter tube. The number of grain indentation pluralities along length 830 is represented by location 819n, with 819a being the location of a first grain plurality, 819b being the location of a second grain plurality, and so on. Each grain plurality may be separated by a space 829 along length 830, but in other embodiments the pluralities of grain indentations may be continuously spaced. The amount of light scattered is represented by 824n, with 824a representing a first amount of light scattered at position 819a, 824b representing a second amount of light scattered at position 819b, and so on. The light scattered 824n at location 819n is the light scattered 824 divided by one minus the product of the light scattered 824n and the location 819n of each grain plurality. A graphical representation of this formula is as follows:

$$Light scattering strength at position n \text{:} \frac{{s}_{n}}{1 - {s}_{n} \cdot n} \qquad$$

In one embodiment, the transparent fiber optic portion may be about 800 mm in length, where about 25 pluralities of grain indentations are disposed along the transparent fiber optic surface from the first position to the second position to provide for a homogenous scattering of light along the length of the fiber optic. In each plurality of grain indentations, two or more grain indentations are disposed proximate to each other.

FIGS. 9a and 9b graphically depict FIG. 8's ratio of scattered light intensity per grain plurality group on an X-Y axis. The percentage 924 of light scattered from the initial light intensity is represented by the Y-axis, while each point on the X-axis 919 represents a single grain plurality group. FIG. 9a is represents the percentage of initial light intensity scattered from 0 to 50 grain plurality groups, while FIG. 9b depicts a subset of FIG. 9a, representing the percentage of initial light intensity scattered from 0 to 25 grain plurality groups. For example, FIG. 9b illustrates that about 2.0% of the initial light intensity is scattered at the first grain plurality group, while about 2.5% of the initial light intensity is scattered at about the tenth grain plurality group.

While particular elements, embodiments, and applications of the present invention have been shown and described, it is understood that the invention is not limited thereto because modifications may be made by those skilled in the art, particularly in light of the foregoing teaching. It is therefore contemplated by the appended claims to cover such modifications and incorporate those features which come within the spirit and scope of the invention.

1. A catheter system for homogeneous trans-illumination of a surgical area, the system comprising:
a catheter tube having a distal end and a proximal end, the distal end comprising a transparent fiber optic portion;
said transparent fiber optic portion comprising a fiber optic configured to transmit light, a proximal end, a distal end, a surface that establishes an outer circumference of the fiber optic portion, and a plurality of grain indentation groups, each grain indentation group comprised of two or more individual grain indentations having a depth below the surface of the fiber optic portion that communicate light through the outer surface of the fiber optic portion;
wherein the plurality of grain indentation groups are disposed along the surface from a first position proximate to the proximal end of the transparent fiber optic portion to a second position proximate to the distal end of the transparent fiber optic portion, and
wherein a size of the grain indentations increases in a direction from the first position to the second position.

2. The system of claim 1, wherein the grain indentations increase in size continuously from the first position to the second position.

3. The system of claim 1, wherein the grain indentations increase in size in a groupwise manner between neighboring indentation groups.

4. The system of claim 1, wherein the plurality of grain indentation groups is in a repeating pattern along the surface from the first position to a second position.

5. The system of claim 4, wherein the repeating pattern comprises connected indentations.

6. The system of claim 1, wherein the grain indentation are disposed below the surface a distance less than or equal to the diameter of the grain indentations.

7. The system of claim 1, wherein the surface of the transparent fiber optic portion further comprises radiopaque and non-radiopaque markings.

8. The system of claim 1, wherein the proximal end of the catheter tube is defined by a non-transparent portion.

9. The system of claim 1, wherein the transparent fiber optic portion and the proximal end of the catheter tube are detachably connected.

10. The system of claim 1, wherein the transparent fiber optic portion is about 800 mm in length, wherein the plurality of grain indentations are dispersed in 25 groups disposed along the transparent fiber optic surface from the first position to the second position, wherein two or more of the individual grain indentations are disposed at a distance from each other that is shorter than a distance between two or more of the plurality of grain indentation groups.

11. The system of claim 10, wherein adjacent pluralities of grain indentation groups are equally spaced from each other from the first position to the second position.

12. The system of claim 10, wherein the pluralities of grain indentation groups are continuously spaced from the first position to the second position.

13. A catheter system for homogeneous trans-illumination of a surgical area, the system comprising:
a catheter tube having a distal end and a proximal end, the distal end comprising a transparent fiber optic portion;
said transparent fiber optic portion comprising a fiber optic configured to transmit light, a proximal end, a distal end, a surface that establishes an outer circumference of the fiber optic portion, and a plurality of grain indentation groups, each grain indentation group comprised of two or more individual grain indentations having a depth below the surface of the fiber optic portion that communicate light through the outer surface of the fiber optic portion;
wherein the plurality of grain indentation groups are disposed along the surface from a first position proximate to the proximal end of the transparent fiber optic portion to a second position proximate to the distal end of the transparent fiber optic portion, and
wherein a density of the grain indentations increases in a direction from the first position to the second position.

14. The system of claim 13, wherein a first group of indentations most proximate to the proximal end of the fiber optic portion has a density lower than a second group of indentations that is disposed closer to the distal end of the fiber optic portion than the first group of indentations.

15. The system of claim 13, wherein the plurality of grain indentation groups are disposed in a repeating pattern along the surface from the first position to the second position.

16. The system of claim 15, wherein the repeating pattern comprises a plurality of connected indentations.

17. The system of claim 13, wherein the transparent fiber optic portion is about 800 mm in length, wherein the plurality of grain indentations are dispersed in 25 groups disposed along the transparent fiber optic surface from the first position to the second position, wherein two or more of the individual grain indentations are disposed at a distance from each other that is shorter than a distance between two or more of the plurality of grain indentation groups.

18. The system of claim 17, wherein adjacent pluralities of grain indentation groups are equally spaced from each other from the first position to the second position.

19. The system of claim 17, wherein the pluralities of grain indentation groups are continuously spaced from the first position to the second position.

20. The system of claim 13, wherein the grain indentations are disposed below the surface a distance less than or equal to the diameter of the grain indentations.

21. A catheter system for homogeneous trans-illumination of a surgical area, the system comprising:
a catheter tube having a distal end and a proximal end, the distal end comprising a transparent fiber optic portion;
said transparent fiber optic portion comprising a fiber optic configured to transmit light, a proximal end, a distal end, a distal tip, a surface that establishes an outer circumference of the fiber optic, a plurality of grain indentation groups, each grain indentation group comprised of two or more individual grain indentations having a depth below the surface of the fiber optic portion that communicate light through the outer surface of the fiber optic portion, and a first and second reflective coating;
wherein the plurality of grain indentation groups and the first reflective coating are disposed along the surface from a first position proximate to the proximal end of the transparent fiber optic portion to a second position proximate to the distal end of the transparent fiber optic portion,
and wherein the second reflective coating is disposed at the distal tip of the transparent fiber optic portion.

22. The system of claim 21, wherein a size of the grain indentations increases in a direction from the first position to the second position.

23. The system of claim 21, wherein a density of the grain indentations increases in a direction from the first position to the second position.

24. The system of claim 21, wherein the depth of the grain indentations are from 10 to 100 microns, and the diameter of the grain indentations are from 10 to 100 microns.
